(19) United States
(12) Patent Application Publication
(10) Pub. No.: US 2022/0401168 A1
(43) Pub. Date: Dec. 22, 2022

(54) Slave Device and Control Method Therefor, and Eye Surgery Device and Control Method Therefor

(71) Applicants: Korea Advanced Institute of Science and Technology, Daejeon (KR); Easyendo Surgical, Inc., Daejeon (KR)

(72) Inventors: Dong Soo KWON, Daejeon (KR); Un Je YANG, Daejeon (KR); Duk Yoo KONG, Daejeon (KR); Joon Hwan KIM, Daejeon (KR); Duk Sang KIM, Daejeon (KR); Jeong Do AHN, Daejeon (KR); Chang Kyun KIM, Daejeon (KR); Byung Sik CHEON, Daejeon (KR)

(21) Appl. No.: 17/621,558

(22) PCT Filed: Jun. 19, 2020
(86) PCT No.: PCT/KR2020/008018

§ 371 Date: Dec. 21, 2021

(30) Foreign Application Priority Data
Jun. 21, 2019 (KR) 10-2019-0074316
Jun. 21, 2019 (KR) 10-2019-0074351

(51) Int. Cl. A61B 34/37; A61F 9/007; A61B 90/25

(52) U.S. Cl. CPC A61B 34/37; A61F 9/007; A61B 90/25; A61B 2017/00075

(57) ABSTRACT

A slave device according to an example embodiment may comprise: a lower shaft; an upper shaft connected to the lower shaft so as to be able to slide with a single degree of freedom; a lower gripper rotatably supporting the lower shaft; an upper gripper rotatably supporting the upper shaft; a lower delta robot movably supporting the lower gripper; and an upper delta robot movably supporting the upper gripper.

### TECHNICAL FIELD

The following description relates to a slave device and a control method therefor, and an eye surgery device and a control method therefor.

### BACKGROUND ART

A master device and a slave device electrically transmit and receive signals with each other. A user may directly drive the master device, and the slave device may be remotely controlled based on a movement of the master device. For example, the master device and the slave device are used in a surgical field that requires detailed work.

An eye surgery device includes a surgical instrument that penetrates a surface of an eye to be inserted into the eye. There is a need for a technique that does not damage the surface of the eye while the surgical instrument is moved. In addition, an internal region of the eye that may be observed through a pupil thereof is limited, and thus, there is an issue that it is difficult to observe the inside of the eye.

### DISCLOSURE OF THE INVENTION

### Technical Goals

An object of an example embodiment is to provide a slave device and a method of controlling the slave device.

### Technical Solutions

According to an aspect, there is provided a lower shaft; an upper shaft slidably connected to the lower shaft in one degree of freedom; a lower gripper configured to rotatably support the lower shaft; an upper gripper configured to rotatably support the upper shaft; a lower delta robot configured to movably support the lower gripper; and an upper delta robot configured to movably support the upper gripper.

The lower shaft may be configured to maintain a position relative to the lower gripper irrespective of a change in a distance between the lower gripper and the upper gripper in an axial direction of the lower shaft.

Each of the lower shaft and the upper shaft may be rotatably supported in two degrees of freedom by the lower gripper and the upper gripper.

Each of the lower delta robot and the upper delta robot may include three support rods; three movement parts, each of the three movement parts configured to move in a longitudinal direction of each of the three support rods; and three arms connecting the three movement parts and a gripper.

Each of the lower delta robot and the upper delta robot may further include three guide rods arranged in parallel with the three support rods and guiding movements of the three movement parts.

The three support rods of the lower delta robot may be in parallel with the three support rods of the upper delta robot.

The three support rods of the lower delta robot may be separated from the three support rods of the upper delta robot.

The slave device may further include a surgical instrument including a surgical tip having a smaller thickness than the lower shaft and a rotation module which is placed at a lower end of the lower shaft and configured to rotate the surgical tip.

A distance between the lower gripper and the upper gripper may be adjusted while the surgical instrument maintains a position separated from the lower gripper in an axial direction of the lower shaft.

According to another aspect, there is provided a method of controlling a slave device including a lower shaft, an upper shaft slidably connected to the lower shaft in one degree of freedom, a lower gripper configured to rotatably support the lower shaft, an upper gripper configured to rotatably support the upper shaft, and a surgical instrument provided at a lower end of the lower shaft, and the method may include determining a remote rotation center of the surgical instrument; receiving a target point of a tip of the surgical instrument; determining a reaching point of the lower gripper for placing the tip of the surgical instrument at the target point based on the remote rotation center and the target point of the tip of the surgical instrument; determining a reaching point of the upper gripper based on the remote rotation center and the reaching point of the lower gripper; and moving the lower gripper to the reaching point of the lower gripper and moving the upper gripper to the reaching point of the upper gripper in a state in which at least one point of the surgical instrument is set to pass through the remote rotation center.

The method of controlling the slave device may further include determining whether the tip of the surgical instrument is able to reach the target point.

The method of controlling the slave device may further include determining whether the tip of the surgical instrument is able to reach the target point based on a size of a surgical operation object.

The method of controlling the slave device may further include calculating a position closest to the target point based on a state in which a distance between the lower gripper and the upper gripper is shortest, if the tip of the surgical instrument is determined to be unable to reach the target point.

In the determining of the reaching point of the upper gripper, the reaching point of the upper gripper may be determined on an imaginary extension line passing through the remote rotation center and the reaching point of the lower gripper.

In the determining of the reaching point of the upper gripper, a point that is in a movable region of the upper gripper and separated by the longest distance from the lower gripper may be determined as the reaching point of the upper gripper.

According to another aspect, there is provided an eye surgery device including a support frame; a first slave device connected to one end of the support frame; a second slave device connected to the other end of the support frame; and a microscope module placed between the first slave device and the second slave device and capable of moving on the support frame.

Each of the first slave device and the second slave device may include a lower shaft; an upper shaft slidably connected to the lower shaft in one degree of freedom; a lower gripper rotatably supporting the lower shaft; an upper gripper rotatably supporting the upper shaft; a lower delta robot movably supporting the lower gripper; an upper delta robot movably supporting the upper gripper; and a surgical instrument provided at a lower end of the lower shaft and capable of penetrating the eye.

Each of the lower delta robot and the upper delta robot may include three support rods; three movement parts that move in a longitudinal direction of the three support rods;

and three arms connecting the three movement parts to the gripper.

The eye surgery device may further include a controller configured to detect a position of a surgical instrument of each of the first slave device and the second slave device and control a position of the microscope module based on the position of the surgical instrument.

According to another aspect, there is provided a method of controlling an eye surgery device including a support frame, a first slave device connected to one end of the support frame and configured to drive a first surgical instrument, a second slave device connected to the other end of the support frame and configured to drive a second surgical instrument, and a microscope module placed between the first slave device and the second slave device, and the method may include receiving rotation amount information of an eye from a master device; setting, on a surface of the eye, an initial remote rotation center of each of the first surgical instrument and the second surgical instrument; calculating a target remote rotation center of each of the first surgical instrument and the second surgical instrument based on the rotation amount information of the eye; and moving the remote rotation center of the first surgical instrument from the initial remote rotation center to the target remote rotation center and moving the remote rotation center of the second surgical instrument from the initial remote rotation center to the target remote rotation center, on the surface of the eye.

The rotation amount information of the eye may include first rotation amount information on a rotation about a first rotation axis passing through a center of the eye, and second rotation amount information on a rotation about a second rotation axis that passes through the center of the eye and is orthogonal to the first rotation axis.

The method of controlling the eye surgery device may further include generating a first movement speed profile required while the first surgical instrument reaches the target remote rotation center from the initial remote rotation center in a state in which a distance between the remote rotation center of the first surgical instrument and the remote rotation center of the second surgical instrument is maintained; and generating a second movement speed profile required while the second surgical instrument reaches the target remote rotation center from the initial remote rotation center in a state in which the distance between the remote rotation center of the first surgical instrument and the remote rotation center of the second surgical instrument is maintained.

The calculating of the target remote rotation center of each of the first surgical instrument and the second surgical instrument may include setting a spherical coordinate system based on a center of the eye; calculating, on the spherical coordinate system, an angular change from the initial remote rotation center of the first surgical instrument to the target remote rotation center the first surgical instrument; and calculating, on the spherical coordinate system, an angular change from the initial remote rotation center of the second surgical instrument to the target remote rotation center of the second surgical instrument.

The method of controlling the eye surgery device may further include moving the microscope based on the rotation amount information of the eye.

The method of controlling the eye surgery device may further include moving the microscope based on a target remote rotation center of each of the first surgical instrument and the second surgical instrument.

### Advantageous Effects

A slave device according to an example embodiment may include two delta robots of a three-point support structure arranged in parallel and increases precision by adjusting a distance between upper and lower delta robots or enlarge a work area as needed without changing a position of a surgical instrument.

A slave device according to an example embodiment may receive a desired position of a tip of a surgical instrument received from a master device and drives the surgical instrument while maintaining a remote rotation center, thereby operating the master device intuitively and comfortably without considering the remote rotation center.

According to a method for controlling a slave device of an example embodiment, the slave device may be driven to a remote rotation center based on a movement signal received from a master device, and distances between grippers of upper and lower delta robots may be set as long as possible in order to increase accuracy of the slave device.

According to an eye surgery device and a method for controlling the eye surgery device of an example embodiment, two surgical instruments are moved while maintaining a distance, on a surface of an eye, between portions where the two surgical instruments come into contact with the surface of the eye, and thus, the surface of the eye may not be damaged.

According to an eye surgery device and a method for controlling the eye surgery device of an example embodiment, even when an eye rotates, it is possible to easily observe the inside of the eye by changing a position of a microscope according to a change in a position of a pupil of the eye.

### BRIEF DESCRIPTION OF DRAWINGS

The following drawings attached to the present specification illustrate preferred example embodiments and serve to provide further understanding of the technical idea of the present disclosure together with the detailed description of the present disclosure, and the invention should not be construed as being limited only to the matters described in the drawings.

FIG. 1 is a perspective view illustrating an eye surgery system according to an example embodiment.

FIG. 2 is a perspective view illustrating a slave device and a microscope according to an example embodiment.

FIG. 3 is a perspective view schematically illustrating an internal structure of a slave device according to an example embodiment.

FIG. 4 is a plan view schematically illustrating a surgical instrument according to an example embodiment.

FIG. 5 is a front view schematically illustrating a lower shaft and a surgical instrument according to an example embodiment.

FIG. 6 is a diagram illustrating a relationship between movements of a lower gripper and an upper gripper and movements of a lower shaft and an upper shaft according to the movements of the lower and upper grippers, according to an example embodiment.

FIG. 7 is a front view schematically illustrating a state in which a lower shaft and an upper shaft rotate when a lower gripper and an upper gripper are relatively close to each other, according to an example embodiment.

FIG. 8 is a front view schematically illustrating a state in which a lower shaft and an upper shaft rotate when a lower gripper and an upper gripper are relatively far apart from each other, according to an example embodiment.

FIG. 9 is a block diagram of a slave device according to an example embodiment.

FIG. 10 is a perspective view of a slave device according to an example embodiment.

FIG. 11 is a flowchart illustrating a method of controlling a slave device, according to an example embodiment.

FIGS. 12 and 13 are views schematically illustrating a state in which an eye rotates according to driving of first and second slave devices and a microscope moves according to a rotation of the eye.

FIG. 14 is a flowchart illustrating a method of controlling an eye surgery device, according to an example embodiment.

FIGS. 15 to 17 are plan views illustrating a state in which an eye is rotated by an eye surgery device.

FIG. 18 is a flowchart illustrating operations of calculating a target rotation center of each of first and second surgical instruments, according to an example embodiment.

### BEST MODE FOR CARRYING OUT THE INVENTION

Hereinafter, example embodiments will be described in detail with reference to example drawings. In adding reference numerals to the components of each drawing, it should be noted that the same components are given the same reference numerals as much as possible even though the components are illustrated in different drawings. In addition, in describing the example embodiment, when it is determined that detailed descriptions of a related known configuration or function interferes with understanding of the example embodiment, the detailed descriptions thereof are omitted.

In addition, in describing components of the example embodiment, terms such as first, second, A, B, (a), (b), and so on may be used. The terms are only for distinguishing the components from other components, and attributes, an order, or a sequence of the components are not limited by the terms. When it is described that one component is “connected” or “coupled” to the other component, the component may be directly connected or coupled to the other component, but it will be understood that another component may also be “connected” or “coupled” therebetween.

A component included in one example embodiment and a component having a common function will be described by using the same name in other example embodiments. Unless otherwise stated, descriptions made in one example embodiment may be applied to other example embodiments, and redundant descriptions thereof are omitted.

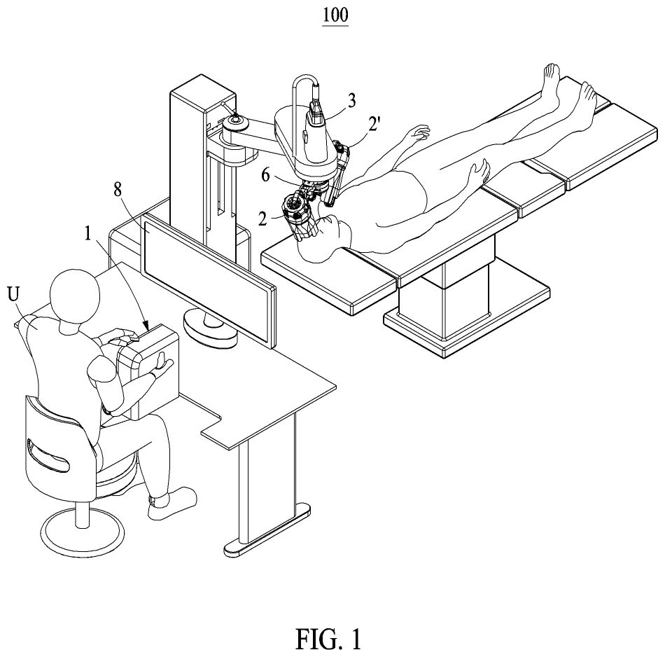

FIG. 1 is a perspective view illustrating an eye surgery system according to an example embodiment.

Referring to FIG. 1, an eye surgery system 100 may be used by a user U to observe on or operate a patient's eye. The eye surgery system 100 may include a master device 1, slave devices 2 and 2′, a microscope 3, a support portion 6, and a display 8.

The master device 1 may generate signals for moving the slave devices 2 and 2′ according to a manipulation of the user U.

The slave devices 2 and 2′ a first slave device 2 that passes through a first portion of a patient's eye to observe on the inside of an eye or operate the eye, and a second slave device 2′ that passes through the second portion of the eye to observe on the inside of the eye or operate the eye. For example, the first portion and the second portion of the eye may be opposite to each other about the pupil of the eye.

The microscope 3 may observe on an eye through the pupil of the eye.

The support portion 6 may support the slave devices 2 and 2′ and the microscope 3.

The display 8 may display an image observed by the microscope 3 and provide the image to the user U in real time.

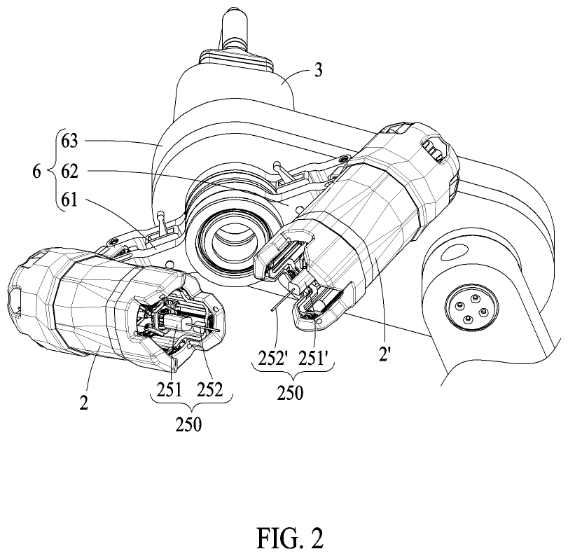

FIG. 2 is a perspective view illustrating the slave devices 2 and 2′ and the microscope according to an example embodiment.

Referring to FIG. 2, the first slave device 2 and the second slave device 2′ may be connected to a lower portion of the support portion 6. The microscope 3 may be connected to an upper portion of the support portion 6. The support portion 6 may include a first support frame 61 for supporting the first slave device 2, a second support frame 62 for supporting the second slave device 2′, and a support base 63 for supporting the microscope 3. For example, the support base 63 may be hinge-connected to be relatively rotatable and may have a plurality of link structures provided in series.

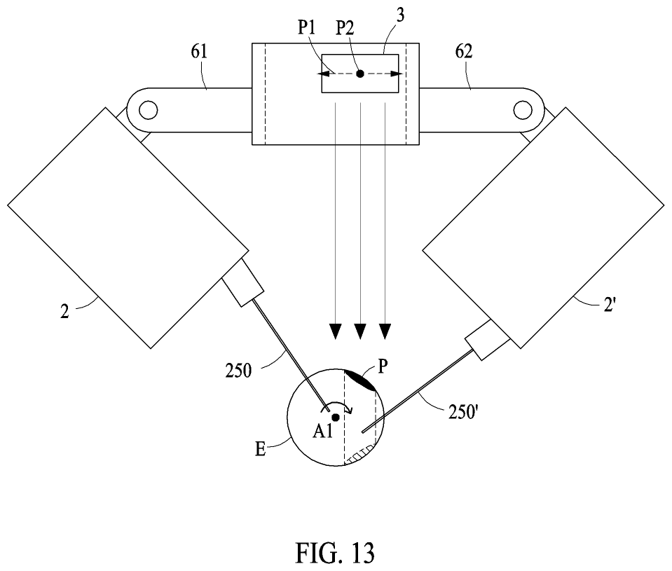

The first support frame 61 and the second support frame 62 may have holes formed through a lower side of the microscope 3, and the microscope 3 may observe on a patient's eye through the holes. The microscope 3 may be movably mounted on the support portion 6. For example, the microscope 3 is movable on the first support frame 61 and the second support frame 62 while maintaining an angle of a lens. The microscope 3 is movable on a plane. For example, the microscope 3 is movable along a first path P1 parallel to the first support frame 61 and a second path P2 perpendicular to the first path P1 (see FIGS. 12 and 13). For example, at least one of the first support frame 61, the second support frame 62, and the support base 63 may accommodate the microscope 3 and provide a movable space for the microscope 3. For example, the support portion 6 may include a first linear actuator (not illustrated) for moving the microscope 3 along the first path P1, and a second linear actuator (not illustrated) for moving the microscope 3 along the second path P2.

The first slave device 2 may include a first surgical instrument 250, and the second slave device 2′ may include a second surgical instrument 250′. The first surgical instrument 250 may include a rotation module 251 and a surgical tip 252 inserted into a patient's eye and rotated by the rotation module 251. The second surgical instrument 250′ may include a rotation module 251′ and a surgical tip 252′ inserted into a patient's eye and rotated by the rotation module 251′. For example, only one of the first surgical instrument 250 and the second surgical instrument 250′ may be inserted into the eye to perform observation or surgery.

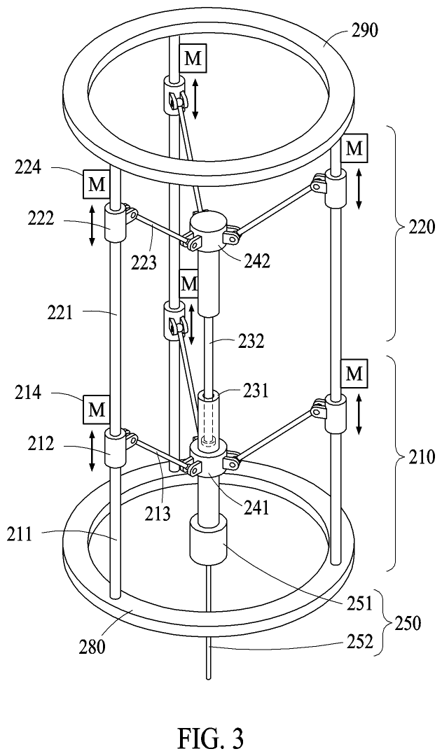

FIG. 3 is a perspective view schematically illustrating an internal structure of a slave device according to an example embodiment.

Referring to FIG. 3, the slave device may include a lower delta robot 210, an to upper delta robot 220, a lower shaft 231, an upper shaft 232, a lower gripper 241, an upper gripper 242, a surgical instrument 250, a lower frame 280, and an upper frame 290.

The lower delta robot 210 may movably support the lower gripper 241. The upper delta robot 220 may movably support the upper gripper 242. The lower delta robot 210 and the upper delta robot 220 may respectively include three support rods 211 and three support rods 221, three movement parts 212 and three movement parts 222 that respectively move along longitudinal directions of the support rods 211 and 221, three arms 213 and three arms 223 that respectively connect the movement parts 212 and the movement parts 222 to the lower grippers 241 and the upper grippers 242, and three drive sources 214 and three drive sources 224 that respectively provide power for moving the three movement parts 212 and the three movement parts 222. The lower delta robot 210 and the upper delta robot 220 may be driven according to a linear actuator method and may perform a precise movement with little vibration and backlash. The three support rods 211 and 221 may be arranged between the lower frame 280 and the upper frame 290.

The arms 213 and 223 may be rotatably connected to the corresponding movement parts 212 and 222, and the arms 213 and 223 may be relatively rotatably connected to the corresponding grippers 241 and 242.

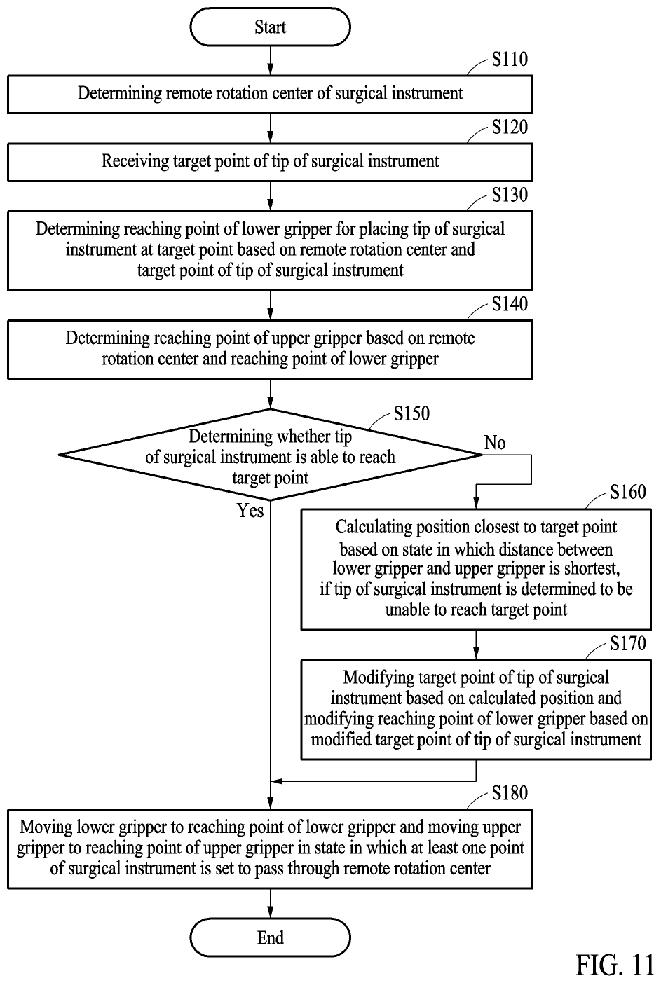

The support rods 211 of the lower delta robot 210 may be parallel to the support rods 221 of the upper delta robot 220. For example, each of the support rods 211 of the lower delta robot 210 and each of the support rods 221 of the upper delta robot 220 may be respectively a lower portion of any one support rod and an upper portion of any one support rod. In other words, the support rods 211 of the lower delta robot 210 may be respectively bonded to the support rods 221 of the upper delta robot 220 without boundaries. According to this structure, only three support rods may guide six movement parts, and thus, the structure may be simply designed. Meanwhile, the support rods 211 of the lower delta robot 210 may be separated from the support rods 221 of the upper delta robot 220 (see FIG. 10).

The lower shaft 231 may be driven by the lower delta robot 210. The lower shaft 231 may be rotatably connected to the lower gripper 241 in two degrees of freedom. For example, the lower shaft 231 may include a joint, which is rotatably connected to the lower gripper 241 in two degrees of freedom, for example, a ball joint or a universal joint. The lower shaft 231 may be fixed to the lower gripper 241 at a point in which the joint is placed. One point of the lower shaft 231 may be fixed to the lower gripper 241.

Hereinafter, a point of the lower gripper 241 at which the lower shaft 231 is fixed may be referred to as a central point of the lower gripper 241.

The upper shaft 232 may be driven by the upper delta robot 220. The upper shaft 232 may be rotatably connected to the upper gripper 242 in two degrees of freedom. For example, the upper shaft 232 may include a joint, which is rotatably connected to the upper gripper 242 in two degrees of freedom, for example, a ball joint or a universal joint. The upper shaft 232 may be fixed to the upper gripper 242 at a point in which the joint is placed. One point of the upper shaft 232 may be fixed to the upper gripper 242. Hereinafter, a point of the upper gripper 242 at which the upper shaft 232 is fixed may be referred to as a central point of the upper gripper 242.

The lower shaft 231 and the upper shaft 232 are relatively slidable. For example, while driving the lower delta robot 210 and/or the upper delta robot 220 to change a position of the lower gripper 241 and/or a position of the upper gripper 242, the lower shaft 231 and the upper shaft 232 are slidable in one degree of freedom. The lower shaft 231 is rotatable in two degrees of freedom with respect to the lower gripper 241, and the rotation of the lower shaft 231 is independent of a change in a distance between the lower gripper 241 and the upper gripper 242 in an axial direction of the lower shaft 231. According to this structure, the slave device may move only the upper shaft 232 while the lower shaft 231 is fixed.

For example, any one of the lower shaft 231 and the upper shaft 232 may include a hollow, and the other may include a slider which is slidable while being inserted into the hollow. For example, as illustrated in FIG. 3, the lower shaft 231 may include a hollow accommodating at least a part of the upper shaft 232, and the upper shaft 232 may include a slider which is slidable while being inserted into the hollow of the lower shaft 231. For example, the slider may slide in one degree of freedom while in surface contact with an inner wall of the lower shaft 231.

The lower gripper 241 may rotatably support the lower shaft 231. The lower gripper 241 may be supported by the three arms 213 of the lower delta robot 210, and a position thereof may be changed based on movements of the three movement parts 212.

The upper gripper 242 may rotatably support the upper shaft 232. The upper gripper 242 may be supported by the three arms 223 of the upper delta robot 220, and a position thereof may be changed based on movements of the three movement parts 222.

While the surgical instrument 250 maintains a position separated from the central point of the lower gripper 241 in an axial direction (a longitudinal direction) of the lower shaft 231, a distance between the lower gripper 241 and the upper gripper 242 may be adjusted. In this case, the lower gripper 241 is fixed, and the upper gripper 242 moves along a path parallel to the longitudinal direction of the lower shaft 231.

The surgical instrument 250 may include a rotation module 251 and a surgical tip 252. The surgical tip 252 may be a longitudinal member. For example, the surgical tip 252 may be parallel to the lower shaft 231 and upper shaft 232. For example, a central axis of the surgical tip 252 may pass through central axes of the lower shaft 231 and the upper shaft 232. The surgical tip 252 may have a less thickness than the lower shaft 231 and may be inserted into an eye through a surface of the eye. The rotation module 251 may be mounted on the lower shaft 231 and may rotate the surgical tip 252. For example, the surgical tip 252 may rotate about an axis parallel or parallel to a longitudinal axis of lower shaft 231.

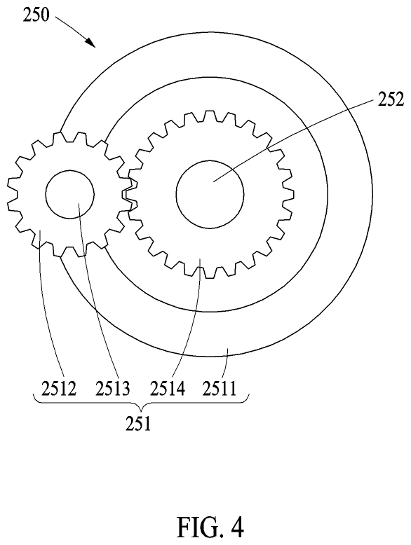

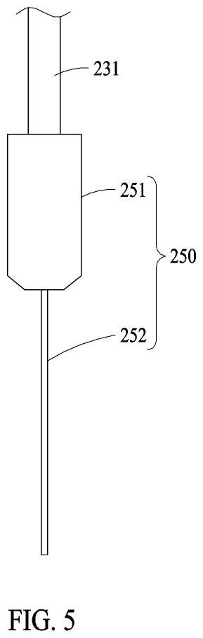

FIG. 4 is a plan view schematically illustrating a surgical instrument according to an example embodiment, and FIG. 5 is a front view schematically illustrating a lower shaft and a surgical instrument according to an example embodiment. FIG. 4 illustrates an internal mechanism of the rotation module 251 schematically illustrated in FIG. 5.

Referring to FIGS. 4 and 5, the rotation module 251 may be installed at a lower end of the lower shaft 231. Unlike this, the rotation module 251 may also be installed elsewhere on the lower shaft 231.

The rotation module 251 may include a main body 2511, a first gear 2512 installed on a side of the main body 2511, a gear shaft 2513 for rotating the first gear 2512, and a second gear 2514 meshing with the first gear 2512. The surgical tip 252 may be rotated along with a rotation of the second gear 2514. For example, a rotation axis of the second gear 2514 may be parallel to or coincident with a central axis of the lower shaft 231.

A drive source installed in the rotation module 251 first rotates the gear shaft 2513, and thus, the first gear 2512 rotates the second gear 2514, and then the surgical tip 252 rotates. Accordingly, a yaw rotation in which a longitudinal direction of the lower shaft 231 is used as a gear axis may be achieved.

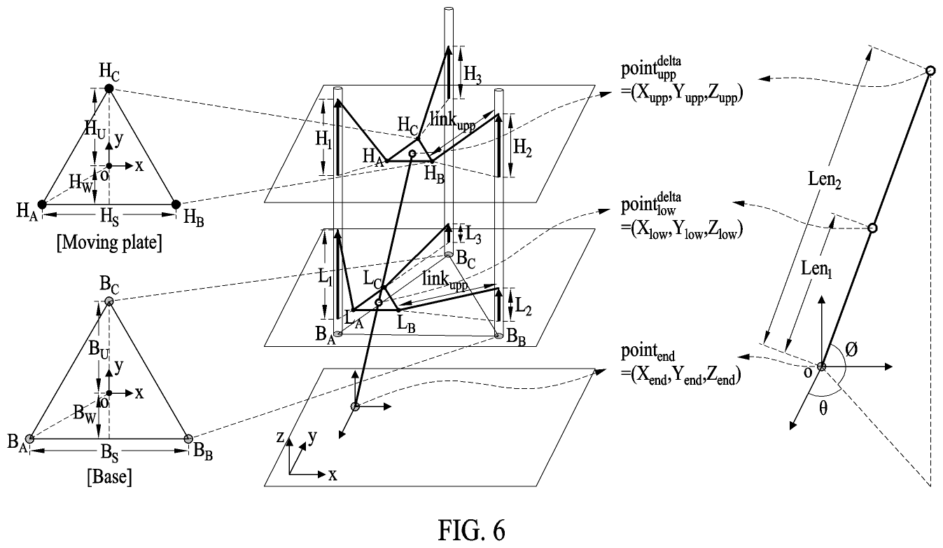

FIG. 6 is a diagram illustrating a relationship between movements of a lower gripper and an upper gripper and movements of a lower shaft and an upper shaft according to the movements of the lower and upper grippers, according to an example embodiment.

Inverse kinematics, forward kinematics, and Jacobian of a dual delta structure constituting a slave device are described in detail with reference to FIG. 6. The dual delta robot uses two delta robots (a lower delta robot and an upper delta robot) that move only in an x-axis direction, a y-axis direction, and a z-axis direction by connecting the two delta robots to each other with a passive joint.

Variables used in the kinematics of the double delta structure are defined as follows.

pointᵤₚₚ^{delta}(Xᵤₚₚ,Yᵤₚₚ,Zᵤₚₚ) orthocenter of moving plate of Upper delta
point_{low}^{delta}(X_{low},Y_{low},Z_{low}) orthocenter of moving plate of Lower delta
point_{end}^{delta}(X_{end},Y_{end},Z_{end}) end point of surgical instrument
linkᵤₚₚ, link_{low} link length of Upper/Lower delta
H_{A}, H_{B}, H_{C}, L_{A}, L_{B}, L_{C} vertex of moving plate of Upper/Lower delta
B_{A}, B_{B}, B_{C} vertex of Base structure
H₁, H₂, H₃, L₁, L₂, L₃ joint displacement of Upper/Lower delta
Len₁ distance from end point of surgical instrument to orthocenter of Lower delta
Len₂ distance from end point of surgical instrument to orthocenter of Upper delta
B_{N} length of edge of Base structure
H_{S}, L_{S} length of edge of Upper/Lower Moving Plate
B_{w} distance from orthocenter of Base structure to edge
H_{w}, L_{w} distance from orthocenter of Upper, Lower Moving Plate to edge
B_{U} distance from orthocenter of Base structure to vertex
H_{U}, L_{U} distance from orthocenter of Upper, Lower Moving Plate to vertex

pointᵤₚₚ^{delta}(Xᵤₚₚ,Yᵤₚₚ,Zᵤₚₚ) is an orthocenter of an upper gripper and indicates a point in which an upper shaft is fixed. The upper shaft is rotatable in two degrees of freedom while one point thereof is fixed to the upper gripper. Here, the orthocenter of the upper gripper indicates an orthocenter of points (H_{A}, H_{B}, H_{C}) in which three arms of an upper delta robot are connected to the upper gripper. In the present application, pointᵤₚₚ^{delta}(Xᵤₚₚ,Yᵤₚₚ,Zᵤₚₚ) is also referred to as a central point of the upper gripper.

point_{low}^{delta}(X_{low},Y_{low},Z_{low}) is an orthocenter of a lower gripper and indicates a point in which a lower shaft is fixed. The lower shaft is rotatable in two degrees of freedom while one point thereof is fixed to the lower gripper. Here, the orthocenter of the lower gripper indicates an orthocenter of points (L_{A}, L_{B}, L_{C}) in which three arms of a lower delta robot are connected to the lower gripper. Because the lower shaft is fixed to the lower gripper, a distance from point_{low}^{delta}(X_{low},Y_{low},Z_{low}) to a surgical instrument may be constant. In the present application, point_{low}^{delta}(X_{low},Y_{low},Z_{low}) is also referred to as a central point of the lower gripper.

linkᵤₚₚ indicates a length of an arm of the upper delta robot, and link_{low} indicates a length of an arm of the lower delta robot. H_{A}, H_{B}, H_{C}, L_{A}, L_{B}, L_{C} indicate points in which each of the upper gripper and the lower gripper is connected to the arm. B_{A}, B_{B}, B_{C} indicate points connected to three support rods of a lower frame. H₁, H₂, H₃, L₁, L₂, L₃ indicate displacements of movement parts of upper and lower delta structures. Len₁ indicates a distance from an end point of a surgical instrument to point_{low}^{delta}(X_{low},Y_{low},Z_{low}). Len₂ indicates a distance from the end point of the surgical instrument to pointᵤₚₚ^{delta}(Xᵤₚₚ,Yᵤₚₚ,Zᵤₚₚ). B_{S} indicates a length between any two of B_{A},B_{B},B_{C} Each of H_{S},L_{S} indicates a length between any two of H_{A},H_{B},H_{C} and a length between any two of L_{A}, L_{B}, L_{C}. B_{w} indicates a distance from an orthocenter of B_{A}, B_{B}, B_{C} to an edge thereof. H_{w},L_{w} respectively indicate a distance from a line connecting any two of H_{A},H_{B},H_{C} to pointᵤₚₚ^{delta}(Xᵤₚₚ,Yᵤₚₚ,Zᵤₚₚ) and a distance from a line connecting any two of L_{A}, L_{B}, L_{C} of the lower gripper to point_{low}^{delta}(X_{low},Y_{low},Z_{low}). B_{U} indicates a distance from an orthocenter of B_{A}, B_{B}, B_{C} to any one ofB_{A}, B_{B}, B_{C}. H_{U},L_{U} respectively indicate a distance from pointᵤₚₚ^{delta}(Xᵤₚₚ,Yᵤₚₚ,Zᵤₚₚ) to any one of H_{A},H_{B},H_{C} and a distance from point_{low}^{delta}(X_{low},Y_{low},Z_{low}) to any one of L_{A}, L_{B}, L_{C}.

Inverse Kinematics

Positions of the lower and upper grippers are determined through a spherical coordinate system based on rotation information received from a master device, and displacement of a surgical instrument may be obtained through this. The full inverse kinematics may be calculated by calculating kinematics of a lever and kinematics of a delta robot and then combining the kinematics. In order to adjust a hardware scale that is characteristics of a proposed structure, a distance between an end point (an end portion of a surgical tip) of the surgical instrument and the upper gripper is referred to as a variable Lenz.

In a first operation, central positions of the lower and upper gripper are obtained from a position of the end point of the surgical instrument by using the kinematics of the lever. Here, it is assumed that the position and an inclination value of the end point of the surgical instrument are given from the master device. Len₂ is assumed to be a constant. In a second operation, the positions of the lower and upper grippers are calculated by using kinematics of a double delta structure.

Kinematics of a lever structure shows a relationship between an end point point_{end}^{delta}(X_{end},Y_{end},Z_{end}) of the surgical instrument, a central point, point_{low}^{delta}(X_{low},Y_{low},Z_{low}) of the lower gripper, and a central point pointᵤₚₚ^{delta}(Xᵤₚₚ,Yᵤₚₚ,Zᵤₚₚ) of the upper gripper. Len₂ indicates a distance from the end point of the surgical instrument to the central point of the upper gripper and is used as a variable for adjusting the hardware scale. Φ indicates a pitch axis azimuth, and Θ indicates a roll axis azimuth.

The azimuth expressed in a spherical coordinate system is expressed in the following form by a Cartesian coordinate system.

$${X}_{upp} = {X}_{end} + {Len}_{2} \cdot \text{?} \cdot cos ( θ ) \qquad ( 1 )$$

$${Y}_{upp} = {Y}_{end} + {Len}_{2} \cdot \text{?} \cdot sin ( θ ) \qquad ( 2 )$$

$${Z}_{upp} = {Z}_{end} + {Len}_{3} \cdot \text{?} \qquad ( 3 )$$

$${X}_{low} = {X}_{end} + {Len}_{1} \cdot \text{?} \cdot cos ( θ ) \qquad ( 4 )$$

$${Y}_{low} = {Y}_{end} + {Len}_{1} \cdot \text{?} \cdot sin ( θ ) \qquad ( 5 )$$

$${Z}_{low} = {Z}_{end} + {Len}_{1} \cdot \text{?} \qquad ( 6 )$$

$$\begin{matrix}{Len}_{2} = \sqrt{\text{?} + {( {Y}_{upp} - {Y}_{end} )}^{2} + \text{?}} \\ ϕ = arccos ( \frac{{Z}_{upp} - {Z}_{end}}{{Len}_{2}} ) \\ θ = arc tan ( \frac{{Y}_{upp} - {Y}_{end}}{{X}_{upp} - {X}_{end}} )\end{matrix}$$

$$\text{?} \text{indicates text missing or illegible when filed}$$

By using a principle of a lever, a central point of a lower delta robot may be obtained through X_{end},Y_{end},Z_{end}, Ø, θ of a tip of a surgical instrument and Len₁ that is a distance between the tip of the surgical instrument and a lower gripper. Len₁ is a constant value that does not change because Len₁ is a distance determined by a length of the mounted surgical instrument. Likewise, a central point of the upper gripper may be obtained through Len₂ that is a distance between the tip of the surgical instrument and the upper gripper.

Next, a relationship between the central points of the upper and lower grippers to and a prismatic joint is obtained through kinematics of a delta robot as follows. The previously determined central point of each delta determines values of six prismatic joints which are H₁, H₂, H₃, L₁, L₂, L₃. The relationship between the central points of the upper and lower grippers and the prismatic joint is as follows.

$${X}_{upp}^{3} + {Y}_{upp}^{3} + {Z}_{upp}^{3} + {a}_{upp}^{2} + {b}_{upp}^{2} + 2 \cdot {a}_{upp} \cdot {X}_{upp} + 2 \cdot {b}_{upp} \cdot {Y}_{upp} + 2 \cdot {Z}_{upp} \cdot {H}_{1} + \text{?} - {link}_{upp}^{2} = 0 \qquad ( 7 )$$

$${X}_{upp}^{2} + {Y}_{upp}^{2} + {Z}_{upp}^{2} + {a}_{upp}^{1} + {b}_{upp}^{2} - 2 \cdot {a}_{upp} \cdot {X}_{upp} + 2 \cdot {b}_{upp} \cdot {Y}_{xpp} + 2 \cdot {Z}_{upp} \cdot {H}_{2} + {H}_{2}^{2} - \text{?} = 0 \qquad ( 8 )$$

$${X}_{upp}^{2} + \text{?} + \text{?} + {c}_{upp}^{3} + 2 \cdot {c}_{upp} \cdot \text{?} + 2 \cdot \text{?} \cdot {H}_{3} + {H}_{3}^{2} - {link}_{upp}^{2} = 0 \qquad ( 9 )$$

$${X}_{low}^{3} + {Y}_{low}^{3} + \text{?} + {a}_{low}^{2} + {b}_{low}^{3} + 2 \cdot {a}_{low} \cdot {X}_{low} + 2 \cdot {b}_{low} \cdot {Y}_{low} + 2 \cdot \text{?} \cdot {L}_{1} + {L}_{1}^{2} - \text{?} = 0 \qquad ( 10 )$$

$${X}_{low}^{3} + {Y}_{low}^{3} + {Z}_{low}^{2} + {a}_{low}^{2} + \text{?} - 2 \cdot {a}_{low} \cdot {X}_{low} + 2 \cdot {b}_{low} \cdot {Y}_{low} + 2 \cdot \text{?} \cdot \text{?} + \text{?} - \text{?} = 0 \qquad ( 11 )$$

$${X}_{low}^{3} + {Y}_{low}^{3} + {Z}_{low}^{2} + {c}_{low}^{2} + 2 \cdot {c}_{low} \cdot {Y}_{low} + 2 \cdot {Z}_{low} \cdot \text{?} + \text{?} - {link}_{low}^{3} = 0 \qquad ( 12 )$$

$$Where :$$

$${a}_{upp} = \frac{{R}_{S}}{2} - \frac{{H}_{S}}{2} , {b}_{upp} = {B}_{W} - {H}_{W} , {c}_{upp} = {H}_{U} - {B}_{U} \\ {a}_{low} = \frac{{B}_{S}}{2} - \frac{{L}_{S}}{2} , {b}_{low} = {B}_{W} - {L}_{W} , {c}_{low} = {L}_{U} - {B}_{U}$$

$$\text{?} \text{indicates text missing or illegible when filed}$$

As a result, the values of the prismatic joints of double delta are as follows.

H_{1(1,2)}=−Zᵤₚₚ±\sqrt{linkᵤₚₚ²−(Xᵤₚₚ+aᵤₚₚ)²−(Yᵤₚₚ+bᵤₚₚ)²} (13)

H_{2(1,2)}=−Zᵤₚₚ±\sqrt{linkᵤₚₚ²−(Xᵤₚₚ−aᵤₚₚ)²−(Yᵤₚₚ+bᵤₚₚ)²} (14)

H_{3(1,2)}=−Zᵤₚₚ±\sqrt{linkᵤₚₚ²−(Xᵤₚₚ²)−(Yᵤₚₚ+cᵤₚₚ)²} (15)

L_{1(1,2)}=−Z_{low}±\sqrt{link_{low}²−(X_{low}+a_{low})²−(Y_{low}+b_{low})²} (16)

L_{2(1,2)}=−Z_{low}±\sqrt{link_{low}²−(X_{low}−a_{low})²−(Y_{low}+b_{low})²} (17)

L_{3(1,2)}=−Z_{low}±\sqrt{link_{low}²−(X_{low}²)−(Y_{low}+c_{low})²} (18)

Kinematically, each prismatic joint may have two solutions, and thus, various combinations may occur, but a slave device according to an example embodiment is designed to have only positive solutions through structural constraints.

Forward Kinematics

First, pointᵤₚₚ^{delta}(Xᵤₚₚ,Yᵤₚₚ,Zᵤₚₚ) that is a central point of the upper gripper is obtained as follows. A following equation may be obtained by subtracting Equation (8) from Equation (7).

4·αᵤₚₚ·Xᵤₚₚ+2·Zᵤₚₚ·H₂+H₂²−2·Zᵤₚₚ·H₂−H₂²=0 (19)

This may be expressed in terms of Xᵤₚₚ as follows.

$${X}_{upp} = \frac{{H}_{2} - {H}_{1}}{2 \cdot {a}_{upp}} \cdot {Z}_{upp} + \frac{{H}_{3}^{2} - {H}_{1}^{2}}{4 \cdot {a}_{upp}} \qquad ( 20 )$$

The following equation may be obtained by subtracting Equation (9) from Equation (7) and then inserting Equation (20) thereinto.

$${Y}_{upp} = \frac{( 2 \cdot {H}_{3} - {H}_{1} - {H}_{2} )}{2 \cdot ( {b}_{upp} - {c}_{upp} )} \cdot {Z}_{upp} + \frac{- {H}_{1}^{2} - {H}_{2}^{3} + 2 {H}_{3}^{2} - 2 {a}_{upp}^{2} - 2 {b}_{upp}^{2} + 2 {c}_{upp}^{3}}{4 \cdot ( {b}_{upp} - {c}_{upp} )} \qquad ( 21 )$$

A solution is obtained by expressing Equation (9) in terms of Zᵤₚₚ and using a quadratic equation.

$${Coef}_{1} \cdot {Z}_{upp}^{2} + {Coef}_{2} \cdot {Z}_{upp} + {Coef}_{3} = 0 \qquad ( 22 )$$

$$Where$$

$${Coef}_{1} = \frac{{( {H}_{1} - {H}_{2} )}^{2}}{4 \cdot {a}_{upp}^{2}} = \frac{{( {H}_{1} + {H}_{2} - 2 \cdot {H}_{3} )}^{3}}{4 \cdot {( {b}_{upp} - {c}_{upp} )}^{2}} + 1$$

$${Coef}_{2} = 2 \cdot {H}_{3} + \frac{( 2 \cdot {a}_{upp}^{2} + 2 \cdot {b}_{upp}^{2} - 2 \cdot {c}_{upp}^{2} + {H}_{1}^{2} + \text{?} - 2 \cdot \text{?} ) \cdot ( {H}_{1} + {H}_{2} - 2 \cdot {H}_{3} )}{4 \cdot {( {b}_{upp} - {c}_{upp} )}^{2}} + \frac{( {H}_{1} - {H}_{3} ) \cdot ( {H}_{1}^{2} - {H}_{2}^{2} )}{4 \cdot {a}_{upp}^{2}} - \frac{{c}_{upp} \cdot ( {H}_{1} + {H}_{2} - 2 \cdot {H}_{3} )}{{b}_{upp} - {c}_{upp}}$$

$${Coef}_{3} = \frac{{( 2 \cdot {a}_{upp}^{2} + 2 \cdot {b}_{upp}^{2} - 2 \cdot {c}_{upp}^{2} + {H}_{1}^{2} + {H}_{3}^{2} - 2 \cdot {H}_{3}^{2} )}^{3}}{16 \cdot {( {b}_{upp} - {c}_{upp} )}^{2}} + \frac{{( \text{?} - {H}_{2}^{2} )}^{2}}{16 \cdot {a}_{upp}^{2}} - \frac{{c}_{upp} \cdot ( 2 \cdot {a}_{upp}^{2} - 2 \cdot {b}_{upp}^{2} - 2 \cdot {c}_{upp}^{2} + {H}_{1}^{3} + {H}_{2}^{2} - 2 \cdot {H}_{3}^{2} )}{2 \cdot ( {b}_{upp} - {c}_{upp} )} + {c}_{upp}^{2} + {H}_{3}^{2} - {link}_{upp}^{2}$$

$$\text{?} \text{indicates text missing or illegible when filed}$$

Inserting Equation (23) into Equations (20) and (21),

$${Z}_{upp ( 1 , 2 )} = \frac{- {Coef}_{2} \pm \sqrt{{Coef}_{2}^{3} - 4 \cdot {Coef}_{1} \cdot {Coef}_{3}}}{2 \cdot {Coef}_{1}} \qquad ( 23 )$$

$${X}_{upp ( 1 , 2 )} = \frac{{H}_{2} - {H}_{1}}{2 \cdot {a}_{upp}} \cdot {Z}_{upp ( 1 , 2 )} + \frac{{H}_{2}^{2} - {H}_{1}^{2}}{4 \cdot {a}_{upp}} \qquad ( 24 )$$

$${Y}_{upp ( 1 , 2 )} = \frac{( 2 \cdot {H}_{3} - {H}_{1} - \text{?} )}{2 \cdot ( {b}_{upp} - {c}_{upp} )} \cdot {Z}_{upp ( 1 , 2 )} + \frac{- {H}_{1}^{2} - \text{?} + 2 {H}_{3}^{2} - 2 {a}_{upp}^{3} - 2 {b}_{upp}^{2} + 2 {c}_{upp}^{3}}{4 \cdot ( {b}_{upp} - {c}_{upp} )} \qquad ( 25 )$$

$$\text{?} \text{indicates text missing or illegible when filed}$$

Possible solutions according to Equations (23) to Equation (25) are represented by two sets according to signs thereof. Just like finding a single solution in inverse kinematics, structural constraint conditions are used to have only positive solutions in forward kinematics. When the same method is also applied to the lower gripper, a value of point_{low}^{delta}(X_{low},Y_{low},Z_{low}) may be obtained. Coordinates point_{end}(X_{end},Y_{end},Z_{end}) of the tip of the surgical instrument based on positions of the upper gripper and the lower gripper may be obtained as follows.

$$\begin{matrix}∅ = arccos \frac{{Z}_{upp} - {Z}_{low}}{{Len}_{2} - {Len}_{1}} , & θ = arctan \frac{{Y}_{upp} - {Y}_{low}}{{X}_{upp} - {X}_{low}}\end{matrix} \qquad ( 26 )$$

$${X}_{end} = {X}_{low} - {Len}_{1} \cdot cos ( ∅ ) \cdot cos ( θ ) \qquad ( 27 )$$

$${Y}_{end} = {Y}_{low} - {Len}_{1} \cdot cos ( ∅ ) \cdot sin ( θ ) \qquad ( 28 )$$

$${Z}_{end} = {Z}_{low} - {Len}_{1} \cdot sin ( ∅ ) \qquad ( 29 )$$

Jacobian

A first operation of finding Jacobian of a double delta structure is to perform partial differentiation of an inverse kinematic relation of a lever structure. A state variable is obtained by combining X_{end}′, Y_{end}′, Z_{end}′ that is a speed component of a tip of a surgical instrument based on a Cartesian coordinate system and \dot{Ø}, \dot{θ}, Lėn₂ that is a speed component based on a spherical coordinate system. Through differential inverse kinematic constraints, relationships between Velᵤₚₚ (Xᵤₚₚ′, Yᵤₚₚ′, Zᵤₚₚ′), Vel_{low} (X_{low}′, Y_{low}′,Z_{low}′), X_{end}, Y_{end}′, Z_{end}′, \dot{Ø}, \dot{θ}, Lėn2 are expressed as follows.

Xᵤₚₚ′=X_{end}′−Len₂·sin(Ø)·cos(θ)·\dot{Ø}−Len₂·cos(Ø)·sin(θ)·\dot{θ}+cos(Ø)+cos(θ)·Lėn2 (30)

Yᵤₚₚ′=Y_{end}′−Len₂·sin(Ø)·cos(θ)·\dot{Ø}−Len₂·cos(Ø)·sin(θ)·\dot{θ}+cos(Ø)+cos(θ)·Lėn2 (31)

Zᵤₚₚ′=Z_{end}′+Len₂·cos(Ø)·\dot{Ø}+sin(Ø)·Lėn₂ (32)

X_{low}′=X_{end}′−Len₂·sin(Ø)·cos(θ)·\dot{Ø}−Len₂·cos(Ø)·sin(θ)·\dot{θ} (33)

Y_{low}′=Y_{end}′−Len₂·sin(Ø)·cos(θ)·\dot{Ø}−Len₂·cos(Ø)·sin(θ)·\dot{θ} (34)

Z_{low}′=Z_{end}′=Len₂·cos(Ø)·\dot{θ} (35)

The relationship may be expressed as a matrix and expressed as follows.

$$\begin{matrix}[ \{ \begin{matrix}{Vel}_{upp} \\ {Vel}_{low}\end{matrix} \} ] = [ {A}_{6 \times 6} ] [ \{ \begin{matrix}\dot{{Pos}_{end}} \\ {Ori}_{end}\end{matrix} \} ] \\ [ \begin{matrix}\dot{{X}_{upp}} \\ \dot{{Y}_{upp}} \\ \dot{{Z}_{upp}} \\ \dot{{X}_{low}} \\ \dot{{Y}_{low}} \\ \dot{{Z}_{low}}\end{matrix} ] = [ \text{} \begin{matrix}1 & 0 & 0 & - {Len}_{2} \cdot sin ∅ \cdot cos θ & - {Len}_{2} \cdot cos ∅ \cdot sin θ & cos ∅ \cdot cos θ \\ 0 & 1 & 0 & - {Len}_{2} \cdot sin ∅ \cdot sin θ & {Len}_{2} \cdot cos ∅ \cdot cos θ & cos ∅ \cdot sin θ \\ 0 & 0 & 1 & {Len}_{2} \cdot cos ∅ & 0 & sin ∅ \\ 1 & 0 & 0 & - {Len}_{1} \cdot sin ∅ \cdot cos θ & - {Len}_{1} \cdot cos ∅ \cdot sin θ & 0 \\ 0 & 1 & 0 & - {Len}_{1} \cdot sin ∅ \cdot sin θ & {Len}_{1} \cdot cos ∅ \cdot cos θ & 0 \\ 0 & 0 & 1 & {Len}_{1} \cdot cos ∅ & 0 & 0\end{matrix} ] [ \text{} \begin{matrix}\dot{{X}_{end}} \\ \dot{{Y}_{end}} \\ \dot{\begin{matrix}{Z}_{end} \\ \dot{∅} \\ \dot{θ} \\ \dot{{Len}_{2}}\end{matrix}}\end{matrix} ]\end{matrix}$$

In a second operation, a relationship between speeds of the upper and lower grippers in the Cartesian coordinate system and speeds of the prismatic joints previously calculated through the partial differentiation of the inverse kinematic relation of the delta structure may be found. A relationship between \dot{H}₁ and Xᵤₚₚ′, Yᵤₚₚ′, Zᵤₚₚ′ through a constraint equation of H_{1(1,2)} may be expressed as follows.

H_{2(1,2)}=−Zᵤₚₚ±\sqrt{linkᵤₚₚ²−(Xᵤₚₚ+aᵤₚₚ)²−(Yᵤₚₚ+bᵤₚₚ)^{z}} (36)

(Zᵤₚₚ+H₁)²=linkᵤₚₚ²−(Xᵤₚₚ+aᵤₚₚ)2+(Yᵤₚₚ+bᵤₚₚ)² (37)

In the above equation, partial differentiation of H₁, Xᵤₚₚ, Yᵤₚₚ, Zᵤₚₚ with respect to time is performed.

2·(Zᵤₚₚ+H₁)(Zᵤₚₚ′+H₁)=−2·(Xᵤₚₚ+aᵤₚₚ)·Xᵤₚₚ′−2·(Yᵤₚₚ+bᵤₚₚ)·Yᵤₚₚ′ (38)

2·(Zᵤₚₚ+H₁)·H₁=−2·(Xᵤₚₚ+aᵤₚₚ)·Xᵤₚₚ′−2(Yᵤₚₚ+bᵤₚₚ)·Yᵤₚₚ′−2·(Zᵤₚₚ+H₂)·Zᵤₚₚ′ (39)

Equation (40) may be obtained by dividing Equation (39) by 2·(Zᵤₚₚ+H₁).

$$\text{?} = - \frac{\text{?}}{\text{?}} \cdot \dot{{X}_{upp}} - \frac{\text{?}}{\text{?}} \cdot \dot{{Y}_{upp}} - \frac{\text{?}}{\text{?}} \cdot \dot{{Z}_{upp}} \qquad ( 40 )$$

$$\text{?} \text{indicates text missing or illegible when filed}$$

By calculating speeds of the remaining prismatic joints, a matrix B₆ₓ₆ may be defined as follows.

$$[ \{ \begin{matrix}\dot{{H}_{3 \times 1}} \\ \dot{{L}_{3 \times 1}}\end{matrix} \} ] = [ {B}_{6 \times 6} ] [ \{ \begin{matrix}{Vel}_{upp} \\ {Vel}_{low}\end{matrix} \} ] \qquad ( 41 )$$

$$\begin{matrix}[ \begin{matrix}\dot{{H}_{1}} \\ \dot{{H}_{2}} \\ \dot{{H}_{3}} \\ \dot{{L}_{1}} \\ \dot{{L}_{2}} \\ \dot{{L}_{3}}\end{matrix} ] = [ \text{} \begin{matrix}\begin{matrix}- \frac{( \text{?} + {a}_{upp} )}{( {Z}_{upp} + {H}_{1} )} & - \frac{( \text{?} + {b}_{upp} )}{( {Z}_{upp} + {H}_{1} )} & - 1 \\ - \frac{( {X}_{upp} - {a}_{upp} )}{( {Z}_{upp} + \text{?} )} & - \frac{( {Y}_{upp} + {b}_{upp} )}{( {Z}_{upp} + \text{?} )} & - 1 \\ - \frac{\text{?}}{( {Z}_{upp} + {H}_{3} )} & - \frac{( \text{?} + {c}_{upp} )}{{Z}_{upp} + \text{?} )} & - 1\end{matrix} & \begin{matrix}⋯ & 0 \\ & ⋮\end{matrix} \\ \begin{matrix}⋮ & \\ 0 & ⋯\end{matrix} & \begin{matrix}- \frac{( {X}_{low} + {a}_{low} )}{( {Z}_{low} + {L}_{1} )} & - \frac{( {Y}_{low} + {b}_{low} )}{( {Z}_{low} + {L}_{1} )} & - 1 \\ - \frac{( {X}_{low} - {a}_{low} )}{( {Z}_{low} - \text{?} )} & - \frac{( {Y}_{low} + {b}_{low} )}{( {Z}_{low} + {L}_{2} )} & - 1 \\ - \frac{( {X}_{low} )}{( \text{?} + {L}_{2} )} & - \frac{( {Y}_{low} + {c}_{low} )}{( {Z}_{low} + {L}_{S} )} & - 1\end{matrix}\end{matrix} ] [ \text{} \begin{matrix}\dot{{X}_{upp}} \\ \dot{{Y}_{upp}} \\ \dot{{Z}_{upp}} \\ \dot{{X}_{low}} \\ \dot{{Y}_{low}} \\ \dot{{Z}_{low}}\end{matrix} ] & ( 42 )\end{matrix} \qquad ( 42 )$$

$$\text{?} \text{indicates text missing or illegible when filed}$$

Finally, a Jacobian matrix between a tip of a surgical instrument and an actuator joint may be obtained by multiplying B₆ₓ₆, and A₆ₓ₆ together.

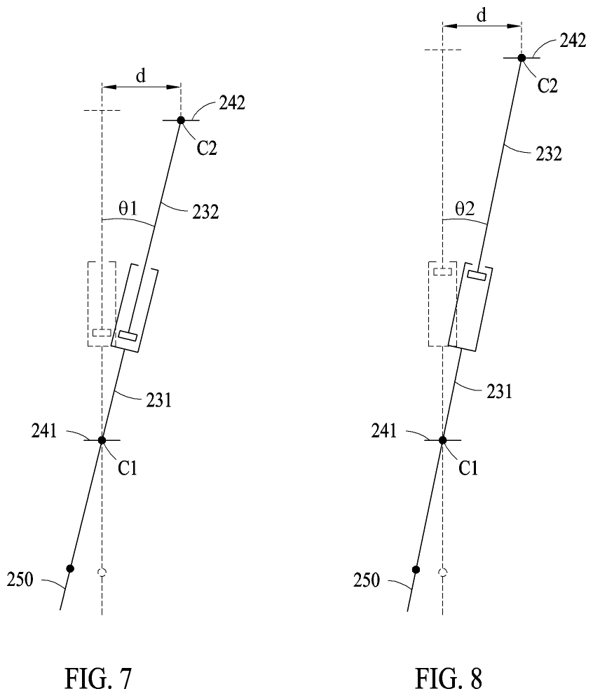

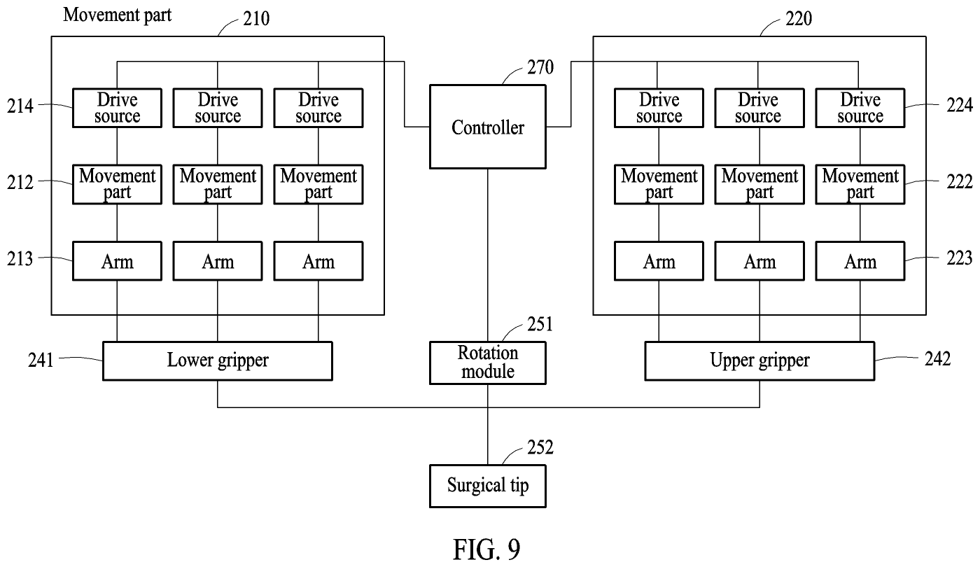

FIG. 7 is a front view schematically illustrating a state in which a lower shaft and an upper shaft rotate when a lower gripper and an upper gripper are relatively close to each other, according to an example embodiment. FIG. 8 is a front view schematically illustrating a state in which a lower shaft and an upper shaft rotate when a lower gripper and an upper gripper are relatively far apart from each other, according to an example embodiment.

Referring to FIGS. 7 and 8, when a distance between the lower gripper 241 and the upper gripper 242 is relatively short (see FIG. 7), an angle is referred to as Θ1 at which the lower gripper 241 and the upper gripper 242 are inclined as the upper gripper 242 moves to the right by a distance d from an initial state in which the lower gripper 241 and the upper gripper 242 are perpendicular to the ground, and when the distance between the lower gripper 241 and the upper gripper 242 is relatively long (see FIG. 8), an angle is referred to as Θ2 at which the lower gripper 241 and the upper gripper 242 are inclined as the upper gripper 242 moves to the right by the distance d from the initial state in which the lower gripper 241 and the upper gripper 242 are perpendicular to the ground, and in this case, Θ1 may be greater than Θ2.

A user may maximize the distance between the lower gripper 241 and the upper gripper 242 within a movable range, thereby increasing precision of a slave device. In addition, even while a distance between a central point C1 of the lower gripper 241 and a central point C2 of the upper gripper 242 is adjusted, a position of the surgical instrument 250 may be fixed to the central point C1 of the lower gripper 241. According to this structure, precision may be adjusted without changing the position of the surgical instrument 250 even while the surgical instrument 250 does a surgery on a part of an eye.

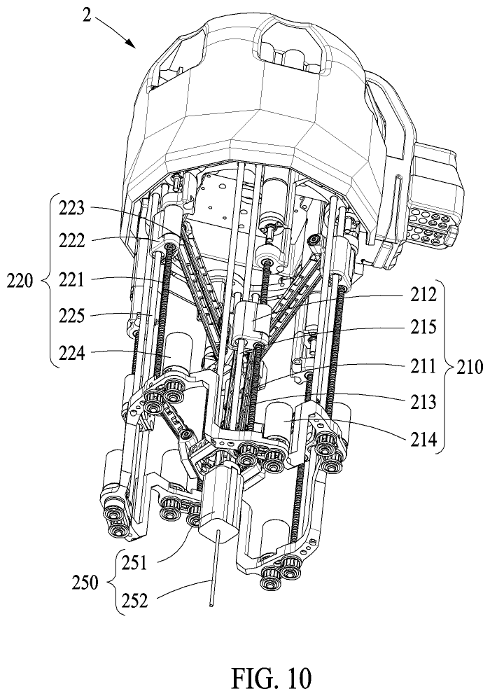

FIG. 9 is a block diagram of a slave device according to an example embodiment.

Referring to FIG. 9, operations of the lower delta robot 210, the upper delta robot 220, and the rotation module 251 are controlled by a controller 270. The lower delta robot 210 controls a position of the lower gripper 241, and the upper delta robot 220 controls a position of the upper gripper 242.

The controller 270 may separately control operations of the three drive sources 214 of the lower delta robot 210. The movement parts 212 move according to the operations of the drive sources 214, and then the arms 213 connected to the movement parts 212 move, thereby moving the lower gripper 241. The lower gripper 241 finally moves a lower shaft.

In addition, the controller 270 may separately control the operations of the three drive sources 224 of the upper delta robot 220. The movement parts 222 move according to the operations of the drive sources 224, and then the arms 223 connected to the movement parts 222 move, thereby moving the upper gripper 242. The upper gripper 242 finally moves an upper shaft.

The surgical tip 252 may move in conjunction with the movement of the lower and upper shafts. The surgical tip 252 may rotate in a total of three degrees of freedom. The controller 270 may control a two-degree-of-freedom rotation of the surgical tip 252 through the lower delta robot 210 and the upper delta robot 220 and control the remaining one-degree-of-freedom rotation through the rotation module 251.

According to the example embodiment described above, a precise movement with less vibration and backlash may be performed by using a robust structure called a delta robot, and by using a double delta robot, a limitation of a small movable range of to the existing delta robot may be overcome.

In addition, by utilizing a precise delta robot structure used throughout the existing industry, precision and an operable range may be adjusted as needed, and thus, the structure may be applied not only to a medical robot, but also to all fields that need to control a precise movement and a wide range of movement as needed.

FIG. 10 is a perspective view of a slave device according to an example embodiment.

Referring to FIG. 10, the lower delta robot 210 and the upper delta robot 220 may respectively include the three support rods 211 and the three support rods 221, the three movement parts 212 and the three movement parts 222, the three arms 213 and the three arms 223, the three drive sources 214 and the three drive sources 224, and three guide rods 215 and three guide rods 225.

The support rods 211 and 221 and the movement parts 212 and 222 may have, for example, a ball-screw linear sliding structure. The drive sources 214 and 224 may cause the movement parts 212 and 222 to move in a longitudinal direction of the support rods 211 and 221 by rotating the support rods 211 and 221. According to this structure, a more precise manipulation may be performed, and a structure resistant to an external impact may be implemented.

The support rods 211 of the lower delta robot 210 may be separated from the support rods 221 of the upper delta robot 220. For example, any one of the support rods 211 of the lower delta robot 210 may be arranged between two adjacent support rods 221 of the upper delta robot 220. In this way, when the support rods 211 of the lower delta robot 210 are separated from the support rods 221 of the upper delta robot 220, movable ranges of the movement parts 212 and 222 may be increased, compared with a state in which the support rods 211 and 221 of the lower delta robot 210 and the upper delta robot 220 are arranged side by side.

The guide rods 215 and 225 are parallel to the support rods 211 and 221 and may guide movements of the movement parts 212 and 222. The movement parts 212 and 222 may move up and down more stably by moving along the guide rods 215 and 225 and the support rods 211 and 221. The guide rods 215 and 225 may increase position control accuracy of the movement parts 212 and 222, and as a result, position control accuracy of the surgical instrument 250 may be increased.

The movement parts 212 and 222 may move along the support rods 211 and 221 and the guide rods 215 and 225 to control a position of the surgical tip 252. The rotation module 251 may rotate the surgical tip 252 about a central axis of the surgical tip 252. A roll, a pitch, and a yaw rotation of the surgical tip 252 may be performed by the movement parts 212 and 222 and the rotation module 251.

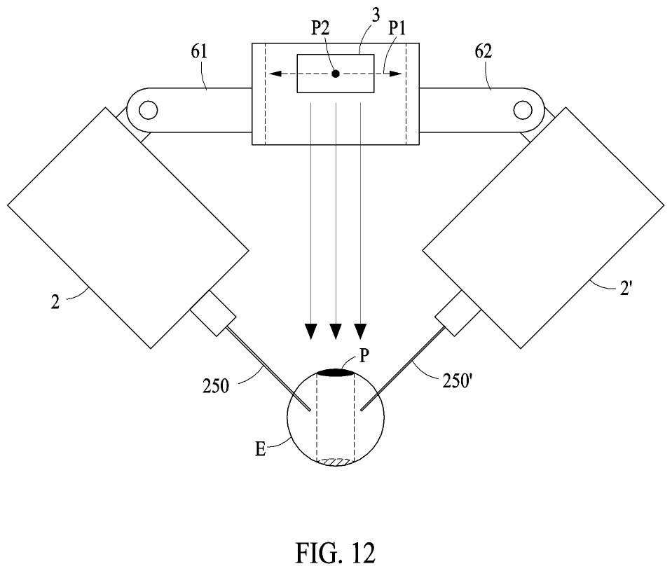

FIG. 11 is a flowchart illustrating a method of controlling a slave device, according to an example embodiment.

Referring to FIG. 11, the method of controlling the slave device may include operation S110 of determining a remote rotation center of the surgical instrument, operation S120 of receiving a target point of a tip of the surgical instrument, operation S130 of determining a reaching point of the lower gripper for placing the tip of the surgical instrument at a target point based on the remote rotation center and the target point of the tip of the surgical instrument, operation S140 of determining a reaching point of the upper gripper based on the remote rotation center and the reaching point of the lower gripper, operation S150 of determining whether the tip of the surgical instrument is able to reach the target point, operation S160 of calculating a position closest to the target point based on a state in which a distance between the lower gripper and the upper gripper is shortest, if the tip of the surgical instrument is determined to be unable to reach the target point, operation S170 of modifying the target point of the tip of the surgical instrument based on the position and modifying the reaching point of the lower gripper based on the modified target point of the tip of the surgical instrument, and operation S180 of moving the lower gripper to the reaching point of the lower gripper and moving the upper gripper to the reaching point of the upper gripper in a state in which at least one point of the surgical instrument is set to pass through the remote rotation center.

In operation S110, a controller may determine a remote rotation center of a surgical instrument. First, the controller moves movement parts of upper and lower delta robots to change positions of lower and upper grippers such that the tip of a surgical instrument comes into contact with a surface of a patient's eye. When the tip of the surgical instrument is in contact with the surface of the eye, the controller may determine a position of the tip of the surgical instrument in the corresponding position as the remote rotation center.

In operation S120, the controller may receive a target point of the tip of the surgical instrument. The target point of the tip of the surgical instrument may be received from the master device 1 (see FIG. 1). The master device 1 may transmit an operation signal to the controller. The controller may operate a slave device based on the operation signal. For example, the target point may be any position in the eye.

In operation S130, the controller may determine a reaching point of the lower gripper for placing the tip of the surgical instrument at the target point, based on the remote rotation center and the target point of the tip of the surgical instrument. The controller may control the position of the surgical tip while maintaining a state in which at least a part of a surgical tip of the surgical instrument passes through the remote rotation center. For example, the surgical tip is a longitudinal member, one point has to pass through the remote rotation center, and when a position (target point) of the tip is determined, a reaching point of the lower gripper may be determined as a single value.

In operation S140, the controller may determine a reaching point of the upper gripper based on the remote rotation center and the reaching point of the lower gripper. Because the reaching point of the lower gripper is determined in operation S130 and the target point of the surgical instrument is determined in operation S120, a position and an angle of a lower shaft are determined, and thus, the reaching point of the upper gripper may be determined as any point of a path parallel to a longitudinal direction of the lower gripper. In other words, the reaching point of the upper gripper may be determined on an imaginary extension line passing through the remote rotation center and the reaching point of the lower gripper. A target point of the tip of the surgical instrument may be determined as any one point as the remote rotation center is determined, and a reaching point of the lower gripper may also be determined as any one position as the position and angle of the surgical instrument are determined. In addition, a position of the upper gripper may be determined as a preset region. The reaching point of the upper gripper in the preset region may be determined as a point separated by the longest distance from the lower gripper. According to this structure, precision of the slave device may be increased (see FIG. 8).

In operation S150, the controller may determine whether the tip of the surgical instrument may reach the target point. For example, when the target point of the tip of the surgical instrument is determined, a position of the lower gripper is determined as any one point, and the lower gripper may not actually reach the corresponding point due to a structural limitation. For example, although the lower gripper may reach the reaching point, the upper gripper may not reach the reaching point due to the structural limitation. As such, if the lower gripper and/or the upper gripper may not reach the reaching point due to the structural limitation, the processing may proceed to operation S160. If the lower gripper and/or the upper gripper may not reach the reaching point, the processing may proceed to operation S180.

In operation S150, the controller may determine whether the tip of the surgical instrument may reach the target point based on a size such as a diameter of, for example, a surgical operation object such as an eye. In other words, by determining that the tip of the surgical instrument may not reach the target point when out of an internal space of the eye even at a position that may be implemented on the slave device, surgical stability may be increased. Here, a boundary surface of the internal space of the eye may be set as, for example, a value received from a user or may also be automatically determined by detecting a diameter of the eye through processing of an image observed through a microscope. For example, when the size of the eye is large, a size of the region reachable by the tip of the surgical instrument may be relatively large.

In operation S160, the controller may calculate a position closest to a target point based on a state in which a distance between the lower gripper and the upper gripper is the shortest. When the distance between the lower gripper and the upper gripper is the shortest, constraints due to a structural limitation of the upper gripper may be reduced. For example, when an initial position of the surgical instrument is referred to as S1 and a target position thereof is referred to as S2, a position closest to the target point may indicate a point closest to S2 on an imaginary line segment region connecting S1 to S2.

In operation S170, the controller may modify the target point of the tip of the surgical instrument based on the position calculated in operation S160 and modify the reaching point of the lower gripper based on the modified target point of the tip of the surgical instrument.

In operation S180, in a state in which at least one point of the surgical instrument is set to pass through a remote rotation center, the controller may move the lower gripper to the reaching point of the lower gripper and move the upper gripper to the reaching point of the upper gripper. While the lower gripper moves from the initial position to the reaching point, the upper gripper may be controlled to adjust an angle of the lower to gripper such that at least one point of the surgical instrument passes through the remote rotation center.

Because the slave device controls the surgical instrument in real time by receiving a signal from the master device in real time, the initial position and the target point of the tip of the surgical instrument may be located substantially adjacent to each other. In a case in which the slave device operates by receiving a signal from the master device in real time at short time intervals, when it is determined in operation S150 that the tip of the surgical instrument is unable to reach the target point, the modified target point of the tip of the surgical instrument may be the same as the target point of the tip of the surgical instrument prior to the modification, and the surgical instrument may not move any more.

FIGS. 12 and 13 are views schematically illustrating a state in which an eye rotates according to driving of first and second slave devices and a microscope moves according to a rotation of the eye.

Referring to FIGS. 12 and 13, the microscope 3 may be placed between the first slave device 2 and the second slave device 2′ and is movable on the support frames 61 and 62. The microscope 3 is movable along two paths P1 and P2 that are orthogonal to each other. The two paths P1 and P2 include a first path P1 and a second path P2 each orthogonal to a certain perpendicular line of a lens of the microscope 3. The microscope 3 is movable on a plane including the first path P1 and the second path P2. For example, the microscope 3 may include two linear actuators that are orthogonal to each other.

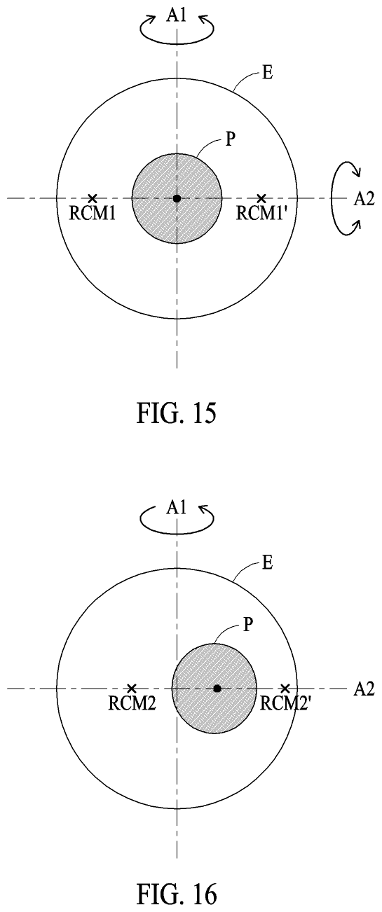

The first slave device 2 and the second slave device 2′ may respectively include a first surgical instrument 250 and a second surgical instrument 250′ which pass through a surface of the eye E. The first slave device 2 and the second slave device 2′ may rotate the eye E by changing angles of the first surgical instrument 250 and the second surgical instrument 250′. The eye E is rotatable about a first rotation axis A1 passing through the center of the eye and is rotatable about a second rotation axis A2 (see FIG. 15) that is perpendicular to the first rotation axis A1 and passes through the center of the eye. The first rotation axis A1 and the second rotation axis A2 may be orthogonal to, for example, an imaginary extension line passing through the center of a pupil P from the center of the eye E.

The microscope 3 may move based on the rotation of the eye E. The microscope 3 may cause a lens to be parallel to the pupil P by moving in response to a change in a position of the pupil P. For example, the amount of movement of the microscope 3 may be determined by the amount of change in a position where the central position of the pupil P is projected onto a movable plane of the microscope 3. According to this method, a region that may be observed inside the eye E may be increased as illustrated in FIGS. 12 and 13.

For example, as illustrated in FIG. 13, the slave devices 2 and 2′ rotate the eye E while relative positions and angles of the surgical instruments 250 and 250′ with respect to the eye E are fixed. According to this control method, a distance between the two surgical instruments 250 and 250′ does not change, and thus, it is possible to prevent the eye E from being damaged during rotation of the eye E.

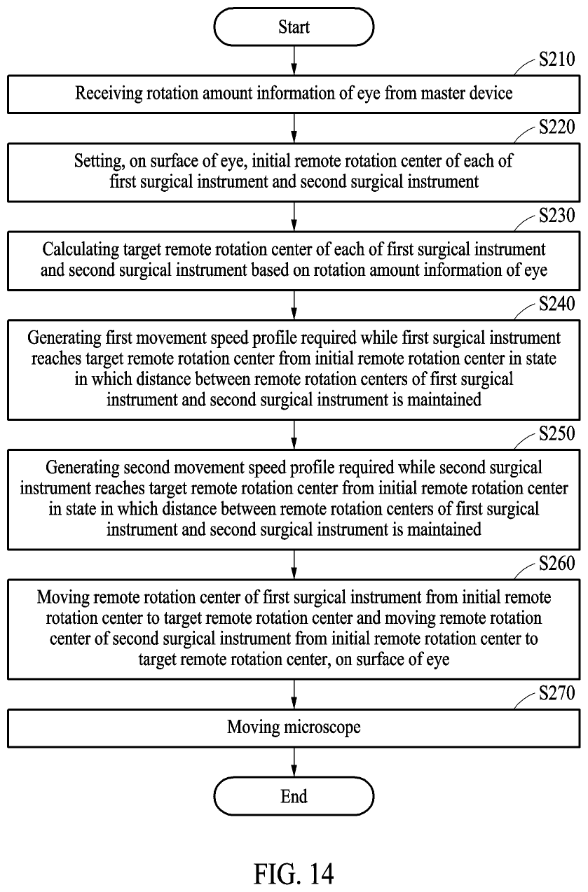

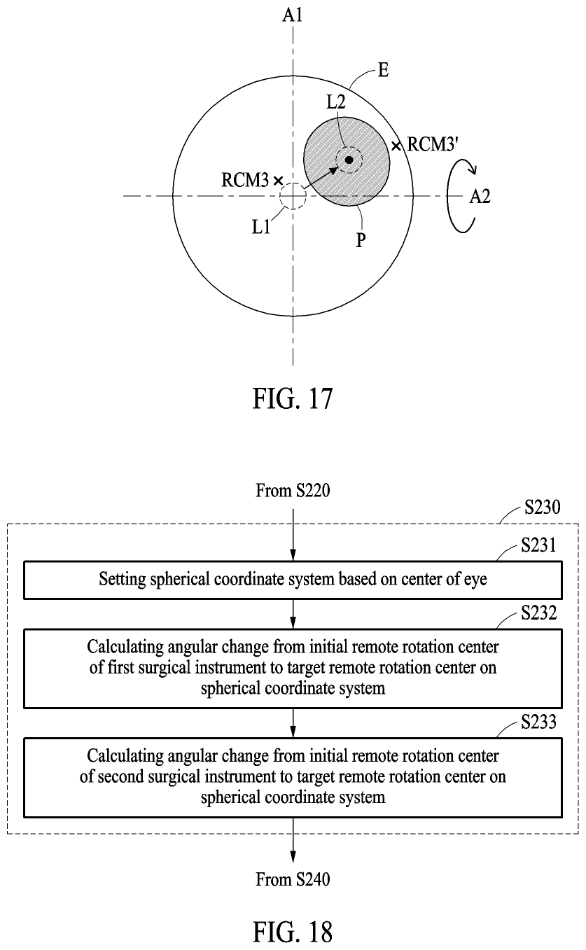

FIG. 14 is a flowchart illustrating a method of controlling an eye surgery device, according to an example embodiment, FIGS. 15 to 17 are plan views illustrating a state in which an eye is rotated by an eye surgery device, and FIG. 18 is a flowchart illustrating operations of calculating a target rotation center of each of first and second surgical instruments, according to an example embodiment.

Referring to FIGS. 14 to 18, a method of controlling an eye surgery device may include operation S210 of receiving rotation amount information of an eye from a master device, operation S220 of setting, on a surface of the eye, an initial remote rotation center of each of the first surgical instrument and the second surgical instrument, operation S230 of calculating a target remote rotation center of each of the first surgical instrument and the second surgical instrument based on the rotation amount information of the eye, operation S240 of generating a first movement speed profile required while the first surgical instrument reaches the target remote rotation center from the initial remote rotation center in a state in which a distance between the remote rotation centers of the first surgical instrument and the second surgical instrument is maintained, operation S250 of generating a second movement speed profile required while the second surgical instrument reaches the target remote rotation center from the initial remote rotation center in a state in which the distance between the remote rotation centers of the first surgical instrument and the second surgical instrument is maintained, operation S260 of moving the remote rotation center of the first surgical instrument from the initial remote rotation center to the target remote rotation center and moving the remote rotation center of the second surgical instrument from the initial remote rotation center to the target remote rotation center, on the surface of the eye, and operation S270 of moving a microscope.

In operation S210, a controller may receive rotation amount information of an eye from a master device. The rotation amount information of the eye may include first rotation amount information on a rotation about a first rotation axis A1 passing through the center of the eye E, and second rotation amount information on a rotation about a second rotation axis A2 that passes through the center of the eye E and is orthogonal to the first rotation axis A1.

In operation S220, the controller may set initial remote rotation centers of a first surgical instrument and a second surgical instrument on a surface of the eye E. For example, when a tip of the first surgical instrument comes into contact with the surface of the eye E, the controller may determine a position of the tip of the first surgical instrument as an initial remote rotation center RCM1. Likewise, when a tip of the second surgical instrument comes into contact with the surface of the eye E, the controller may determine a position of the tip of the second surgical instrument as an initial remote center RCM1′.

In operation S230, the controller may calculate target remote rotation centers RCM3 and RCM3′ of the first surgical instrument and the second surgical instrument, based on rotation amount information of the eye E. Operation S230 may include operation S231 of setting a spherical coordinate system based on the center of the eye E, operation S232 of calculating an angular change from an initial remote rotation center of the first surgical instrument to a target remote rotation center on the spherical coordinate system, and operation S233 of calculating an angular change from an initial remote rotation center of the second surgical instrument to a target remote rotation center on the spherical coordinate system.

In operation S231, the controller may set the spherical coordinate system based on the center of the eye E. The controller may reset the initial remote rotation centers RCM1 and RCM1′ determined to be a Cartesian coordinate system as the spherical coordinate system. The initial remote rotation centers RCM1 and RCM1′ may be represented by two angles.

In operation S232, the controller may calculate an angular change from the initial remote rotation center RCM1 to the target remote rotation center RCM3 of the first surgical instrument on the spherical coordinate system. For example, the remote rotation center may move to RCM2 by rotating from the initial remote rotation center RCM1 by θ about the first rotation axis A1, and in this state, by further rotating by ϕ about the second rotation axis A2, the remote rotation center may move to the target remote rotation center RCM3. The controller may respectively calculate θ and ϕ.

In operation S233, the controller may calculate an angular change from the initial remote rotation center RCM1′ of the second surgical instrument to the target remote rotation center RCM3′ on the spherical coordinate system. The controller may calculate an angular change from the initial remote rotation center RCM1′ of the second surgical instrument to the target remote rotation center RCM3′ on the spherical coordinate system. For example, a remote rotation center may move to RCM2′ by rotating by θ about the first rotation axis A1 from the initial remote rotation center RCM1′, and in this state, the remote rotation center may move to the target remote rotation center RCM3′ by further rotating by ϕ about the second rotation axis A2. The controller may respectively calculate θ and ϕ.

In operation S240, the controller may generate a first movement speed profile required while the first surgical instrument reaches a target remote rotation center from an initial remote rotation center in a state in which a distance between the remote rotation centers of the first and second surgical instruments is maintained. In operation S250, the controller may generate a second movement speed profile required while the second surgical instrument reaches a target remote rotation center from an initial remote rotation center in a state in which the distance between the remote rotation centers of the first and second surgical instruments is maintained. For example, when the eye E rotates in a direction toward the initial remote rotation center RCM1′ of the second surgical instrument about the first rotation axis A1 from the pupil P and then rotates about the rotation axis A2 based on a plan view as illustrated in FIGS. 15 to 17, a distance on a surface of the eye E from the initial remote rotation center RCM1 of the first surgical instrument to the target remote rotation center RCM3 may be longer than a distance on the surface of the eye E from the initial remote rotation center RCM1′ of the second surgical instrument to the target remote rotation center RCM3′. In this case, when a movement speed of the first surgical instrument is faster than a movement speed of the second surgical instrument, a distance between the remote rotation centers of the first surgical instrument and the second surgical instrument may be maintained. In operation S240, the distance between the remote rotation centers of the first and second surgical instruments is maintained, and thus, the eye E may be prevented from being damaged while the remote rotation centers of the first and second surgical instruments changes.

In operation S260, the controller may move the remote rotation center of the first surgical instrument from the initial remote rotation center to the target rotation remote center and may move the remote rotation center of the second surgical instrument from the initial remote rotation center to the target rotation remote center, on a surface of the eye E.

In operation S270, the controller may move a microscope. For example, a position of the microscope may be moved from a first position L1 to a second position L2 as the remote rotation centers of the first surgical instrument and the second surgical instrument are moved. For example, the controller may move the microscope based on rotation amount information of an eye. For example, the controller may control the position of the microscope based on the rotation amount information of the eye received from a master device. In another example, the controller may move the microscope based on the remote rotation centers of the first surgical instrument and the second surgical instrument. For example, the controller may project a position change of the pupil P onto a plane parallel to a plane including the first path P1 (see FIG. 13) and the second path P2 (see FIG. 13) through a change in remote rotation centers of the first surgical instrument and the second surgical instrument, and then set the position of the microscope based on the change in the position of the pupil P in the corresponding plane.

As described above, although example embodiments are described with reference to the limited drawings, those skilled in the art may perform various modifications and changes from the above description. For example, even when the described techniques are performed in a different order from the described method, and/or even when components of the described structure and device are coupled or combined in a different form from the described method or replaced or substituted with other components or equivalents, appropriate results may be achieved.

1. A slave device comprising:
a lower shaft;
an upper shaft slidably connected to the lower shaft in one degree of freedom;
a lower gripper configured to rotatably support the lower shaft;
an upper gripper configured to rotatably support the upper shaft;
a lower delta robot configured to movably support the lower gripper; and
an upper delta robot configured to movably support the upper gripper.

2. The slave device of claim 1, wherein the lower shaft is configured to maintain a position relative to the lower gripper irrespective of a change in a distance between the lower gripper and the upper gripper in an axial direction of the lower shaft.

3. The slave device of claim 1, wherein each of the lower delta robot and the upper delta robot comprises:
three support rods;
three movement parts, each of the three movement parts configured to move in a longitudinal direction of each of the three support rods;
three arms configured to connect the three movement parts and a gripper; and
three guide rods arranged in parallel with the three support rods and configured to guide movements of the three movement parts.

4. The slave device of claim 3, wherein the three support rods of the lower delta robot are provided side by side with and separated from the three support rods of the upper delta robot.

5. The slave device of claim 1, further comprising:
a surgical instrument comprising a surgical tip having a smaller thickness than the lower shaft and a rotation module which is placed at a lower end of the lower shaft and configured to rotate the surgical tip.

6. The slave device of claim 5, wherein a distance between the lower gripper and the upper gripper is adjustable while the surgical instrument maintains a position separated from the lower gripper in an axial direction of the lower shaft.

7. A method of controlling a slave device comprising a lower shaft, an upper shaft slidably connected to the lower shaft in one degree of freedom, a lower gripper configured to rotatably support the lower shaft, an upper gripper configured to rotatably support the upper shaft, and a surgical instrument provided at a lower end of the lower shaft, the method comprising:
determining a remote rotation center of the surgical instrument;
receiving a target point of a tip of the surgical instrument;
determining a reaching point of the lower gripper for placing the tip of the surgical instrument at the target point based on the remote rotation center and the target point of the tip of the surgical instrument;
determining a reaching point of the upper gripper based on the remote rotation center and the reaching point of the lower gripper; and
moving the lower gripper to the reaching point of the lower gripper and moving the upper gripper to the reaching point of the upper gripper in a state in which at least one point of the surgical instrument is set to pass through the remote rotation center.

8. The method of claim 7, further comprising:
determining whether the tip of the surgical instrument is able to reach the target point based on a size of a surgical operation object.

9. The method of claim 8, further comprising:
calculating a position closest to the target point based on a state in which a distance between the lower gripper and the upper gripper is shortest, if the tip of the surgical instrument is determined to be unable to reach the target point.

10. The method of claim 7, wherein, in the determining of the reaching point of the upper gripper, the reaching point of the upper gripper is determined on an imaginary extension line passing through the remote rotation center and the reaching point of the lower gripper.

11. The method of claim 10, wherein, in the determining of the reaching point of the upper gripper, a point that is in a movable region of the upper gripper and separated by the longest distance from the lower gripper is determined as the reaching point of the upper gripper.

12. An eye surgery device for operating on an eye, comprising:
a support frame;
a first slave device connected to one end of the support frame;
a second slave device connected to the other end of the support frame; and
a microscope module placed between the first slave device and the second slave device and capable of moving on the support frame.

13. The eye surgery device of claim 12, further comprising:
a controller configured to detect a position of a surgical instrument of each of the first slave device and the second slave device and control a position of the microscope module based on the position of the surgical instrument.

14. A method of controlling an eye surgery device comprising a support frame, a first slave device connected to one end of the support frame and configured to drive a first surgical instrument, a second slave device connected to the other end of the support frame and configured to drive a second surgical instrument, and a microscope module placed between the first slave device and the second slave device, the method comprising:
receiving rotation amount information of an eye from a master device;
setting, on a surface of the eye, an initial remote rotation center of each of the first surgical instrument and the second surgical instrument;
calculating a target remote rotation center of each of the first surgical instrument and the second surgical instrument based on the rotation amount information of the eye; and
moving the remote rotation center of the first surgical instrument from the initial remote rotation center to the target remote rotation center and moving the remote rotation center of the second surgical instrument from the initial remote rotation center to the target remote rotation center, on the surface of the eye.

15. The method of claim 14,
wherein the rotation amount information of the eye comprises:
first rotation amount information on a rotation about a first rotation axis passing through a center of the eye; and
second rotation amount information on a rotation about a second rotation axis that passes through the center of the eye and is orthogonal to the first rotation axis, and
wherein the method further comprises moving the microscope based on the rotation amount information of the eye.

16. The method of claim 14, further comprising:
generating a first movement speed profile required while the first surgical instrument reaches the target remote rotation center from the initial remote rotation center in a state in which a distance between the remote rotation center of the first surgical instrument and the remote rotation center of the second surgical instrument is maintained; and
generating a second movement speed profile required while the second surgical instrument reaches the target remote rotation center from the initial remote rotation center in a state in which the distance between the remote rotation center of the first surgical instrument and the remote rotation center of the second surgical instrument is maintained.

17. The method of claim 14, wherein the calculating of the target remote rotation center of each of the first surgical instrument and the second surgical instrument comprises:
setting a spherical coordinate system based on a center of the eye;
calculating, on the spherical coordinate system, an angular change from the initial remote rotation center of the first surgical instrument to the target remote rotation center the first surgical instrument; and
calculating, on the spherical coordinate system, an angular change from the initial remote rotation center of the second surgical instrument to the target remote rotation center of the second surgical instrument.
